SEQUENCE LISTING (6 sequences)

SEQ ID NO: 1 - DNA, 40 bp, Synthetic construct
  source 1..40: mol_type = other DNA
cccaggcttggatagcacattcatggcagcgttctttacg

SEQ ID NO: 2 - DNA, 40 bp, Synthetic construct
  source 1..40: mol_type = other DNA
atagatactggatagcacattcatggcagcgttctttacg

SEQ ID NO: 3 - DNA, 32 bp, Synthetic construct
  source 1..32: mol_type = other DNA
tggatagcacattcatggcagcgttctttacg

SEQ ID NO: 4 - DNA, 40 bp, Synthetic construct
  source 1..40: mol_type = other DNA
gatgtataaggcaccgggctcccgctgtttccttgcaata

SEQ ID NO: 5 - DNA, 42 bp, Synthetic construct
  source 1..42: mol_type = other DNA
gacgaccacggtaaacgacgctgtacggagtggtctgtcgtc

SEQ ID NO: 6 - DNA, 26 bp, Synthetic construct
  source 1..26: mol_type = other DNA
ggtggtggtggttgtggtggtggtgg

(19) United States
(12) Patent Application Publication
(10) Pub. No.: US 2025/0388908 A1
(43) Pub. Date: Dec. 25, 2025

(54) METAL HOMEOSTASIS REGULATION

(71) Applicant: Worcester Polytechnic Institute, Worcester, MA (US)

(72) Inventors: Natalie G. Farny, Natick, MA (US); Stephanie Reis, Bellingham, MA (US); Hayley Wigren, Worcester, MA (US)

(21) Appl. No.: 19/248,053

(22) Filed: Jun. 24, 2025

Related U.S. Application Data
(60) Provisional application No. 63/663,865, filed on Jun. 25, 2024.

(51) Int. Cl. C12N 15/115; C12N 15/11; G16B 15/30

(52) U.S. Cl. CPC C12N 15/115; C12N 15/111; G16B 15/30; C12N 2310/16; C12N 2310/531; C12N 2310/532

(57) ABSTRACT

A method of regulating metal homeostasis includes identifying an aptamer having an affinity for a target trace metal, and introducing the aptamer to the target trace metal for sequestering metal ions of the target trace metal to generate a bound aptamer. The bound aptamer may then be directed (injected, ingested, or expunged) around a patient physiology for manipulating the sequestered metal ions based on therapeutic directives. When a harmful metal is to be mediated, the aptamer is directed to a physiological region for sequestering metal ions for removal. When a deficient presence is to be increased, the bound aptamer is directed to a physiological region for increasing the presence of the target trace metal in the physiological region. The trace metals may include metals and metalloids such as iodine, copper, iron, manganese, zinc, selenium, cobalt and molybdenum.

### RELATED APPLICATIONS

This patent application claims the benefit under 35 U.S.C. § 119 (e) of U.S. Provisional Patent App. No. 63/663,865, filed Jun. 25, 2024, entitled “METAL HOMEOSTASIS REGULATION,” incorporated herein by reference in entirety.

### REFERENCE TO AN ELECTRONIC LISTING

The contents of the electronic sequence listing WPI24-20_wipo_st26 seq listing.xml (Size: 6,398 bytes; and Date of Creation: Sep. 3, 2025) is herein incorporated by reference in its entirety.

### BACKGROUND

Trace metals, such as iron, zinc, copper, manganese, and selenium, are essential for healthy development. Inborn or acquired errors of metal metabolism are a collection of diseases in which essential trace metal homeostasis is disrupted, causing negative health consequences. Other metals, such as so-called heavy metals are known for which human contact can be potentially hazardous. Trace metals are moved throughout a healthy organism by metal ion transporters, typically a protein having an affinity for the metal ion. Genetic disorders affecting these proteins lead to an imbalance in the homeostasis of these metal ions, and result in negative health effects from either an excess or deficiency of the metal ions.

### SUMMARY

A method of regulating metal homeostasis includes identifying an aptamer having an affinity for a target trace metal, and introducing the aptamer to the target trace metal for sequestering metal ions of the target trace metal to generate a bound aptamer. The bound aptamer may then be directed (injected, ingested, or expunged) around a patient physiology for manipulating the sequestered metal ions based on therapeutic directives. When a harmful metal is to be mediated, the aptamer is directed to a physiological region for sequestering metal ions for removal. When a deficient presence is to be increased, the bound aptamer is directed to a physiological region for increasing the presence of the target trace metal in the physiological region. The trace metals may include metals and metalloids such as iodine, copper, iron, manganese, zinc, selenium, cobalt and molybdenum, or any suitable metal ion for which a binding aptamer may be generated.

Configurations herein are based, in part, on the observation that human and animal physiology relies on a healthy balance, or homeostasis, of metal ions resulting from metal ion transporters that are part of natural physiology. Such so-called “trace elements” (or trace metals) are minerals present in living tissues in small amounts. Some of them are known to be nutritionally beneficial, others may be essential or nonessential, however an excess or depletion can often trigger health complications. Trace elements function primarily as catalysts in enzyme systems; some metallic ions, such as iron and copper, participate in oxidation-reduction reactions in energy metabolism. Iron, as a constituent of hemoglobin and myoglobin, also plays a vital role in the transport of oxygen, for example.

Unfortunately, conventional approaches to diseases affecting metal ion transport proteins and metal ion homeostasis suffer from the shortcoming that treatments are often directed to masking symptoms rather than curing the cause, and tend to be time critical. For example, copper (Cu) is known to be an “essential trace element”, as it is necessary for specific redox reactions and functions that occur in the human body. However, even though copper is required for these functions, excess copper present in an organism can be toxic and result in negative health impacts. A balance between the two levels is necessary, and thus the human body has designed mechanisms to ensure that the amount of copper present remains in homeostasis. When homeostasis fails due to inborn errors of metabolism, the result is copper metabolism diseases, such as Wilson's disease and Menkes disease. While there are treatments for these diseases that exist, they need to be administered early and often. Most of the therapies that exist are intensive and multi-faceted from surgeries and hemodialysis for Wilson's disease to copper-histidine treatments for Menkes disease, which is a treatment that only addresses symptoms rather than the core of the disease. Other therapies, such as chelators, can reduce excess trace elements, but are non-specific, and would therefore reduce other essential and/or beneficial trace metals which then have to be replaced. Furthermore, treatments must generally be administered throughout the entire life of the patient to prevent irreversible impacts.

Accordingly, configurations herein substantially overcome the shortcomings of conventional ion homeostasis disruption by providing an aptamer based approach for targeting specific metal ions and utilizing a nucleic acid to bind with the aptamer, sequester the targeted metal ion, and promoting either an increase or decrease of metal ion concentration in tissues or regions through the bound aptamer and sequestered metal ion.

Aptamers are short (generally less than 100 nucleotide) stretches of DNA or RNA, or any chemically modified nucleic acid including but not limited to morpholinos, locked nucleic acids (LNAs), left handed (L-enantiomeric, also known as “mirror” DNA or RNA, or Spiegelmers), and may also include modifications such as but not limited to pseudouridines, 2′-F, 2′-OMe, 2′-MOE, phosphorothioated backbones, thio-phosphoroamidated backbones, or other modifications to increase stability, specificity, or immunotolerance. As is well known in the art, DNA (deoxyribonucleic acid) and RNA (ribonucleic acid) are both nucleic acids, which are essential molecules for all known forms of life. Aptamers generally do not encode for protein (like mRNA), or perform specific cellular functions (like tRNA or rRNA).

Development of aptamers is most commonly performed using the Selective Evolution of Ligands by Exponential Enrichment (SELEX) method, as is known in the art, wherein a random pool of oligonucleotides are screened against a particular target molecule. Aptamers can be selected, through SELEX or otherwise generated, to bind with high sensitivity and specificity to a target, which may be any suitable structure from an ion to a small molecule to a cellular protein or component. In configurations herein, aptamers are selected to bind specifically to essential trace metal ions. This binding can be used to sequester the target ion for the purpose of eliminating the ion, or may be used to deliver metal ions to target cells or tissues.

Essential trace metals and metalloids include iodine, copper, iron, manganese, zinc, selenium, cobalt and molybdenum. Hyper accumulation of some of these metals, such as copper, iron, and manganese, in the brain can lead to neurotoxicity. Low levels of these metals and metalloids can render enzymes non-functional and lead to a wide range of metabolic diseases. Inborn errors of metal metabolism are genetic diseases that affect the transport, absorbance, distribution, and secretion of metal ions. Other metals may be harmful at even small levels, such as mercury and lead. While each of these diseases is considered rare, configurations herein may be used to treat these diseases collectively.

In further detail, configurations herein perform metal homeostasis regulation in a human or animal physiology by identifying a binding aptamer having an affinity for a trace metal. The binding aptamer may be introduced into the physiology or combined with other nucleic acids to form a modified nucleic acid. The modified nucleic acid is introduced into a therapeutic region, organ or systemic flow for sequestering the trace metal, and the modified nucleic acid removed from the therapeutic region following sequestering of the trace metal, thereby reducing or eliminating a harmful concentration of the trace metal.

Conversely, a deficiency of a trace metal may also be resolved, by invoking the method for metal homeostasis regulation for identifying a binding aptamer having an affinity for a trace metal and introducing or appending the binding aptamer to a nucleic acid to form a modified nucleic acid as with the removal scenario. However, the trace metal is then appended to the binding aptamer in the modified nucleic acid outside the organism, and the modified nucleic acid introduced into the therapeutic region for transporting the trace metal. In either case, the binding aptamer is itself a nucleic acid, and may be further modified with other nucleic acids to provide an effective delivery strategy. Those nucleic acids from which it is made may be standard nucleic acids or modified nucleic acids.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION

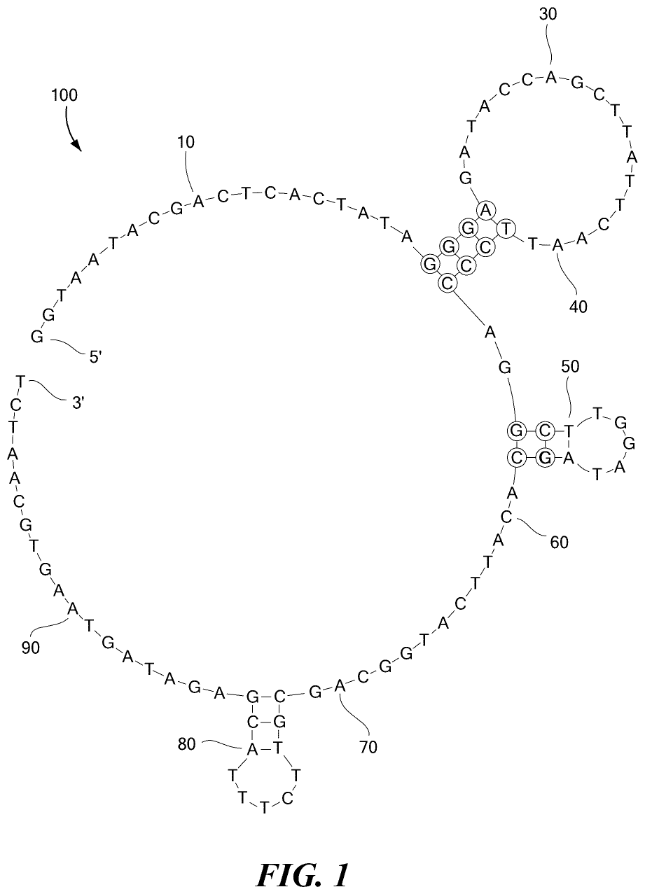

FIG. 1 is a diagram of a binding aptamer demonstrating binding interaction locations suitable for use with configurations herein;

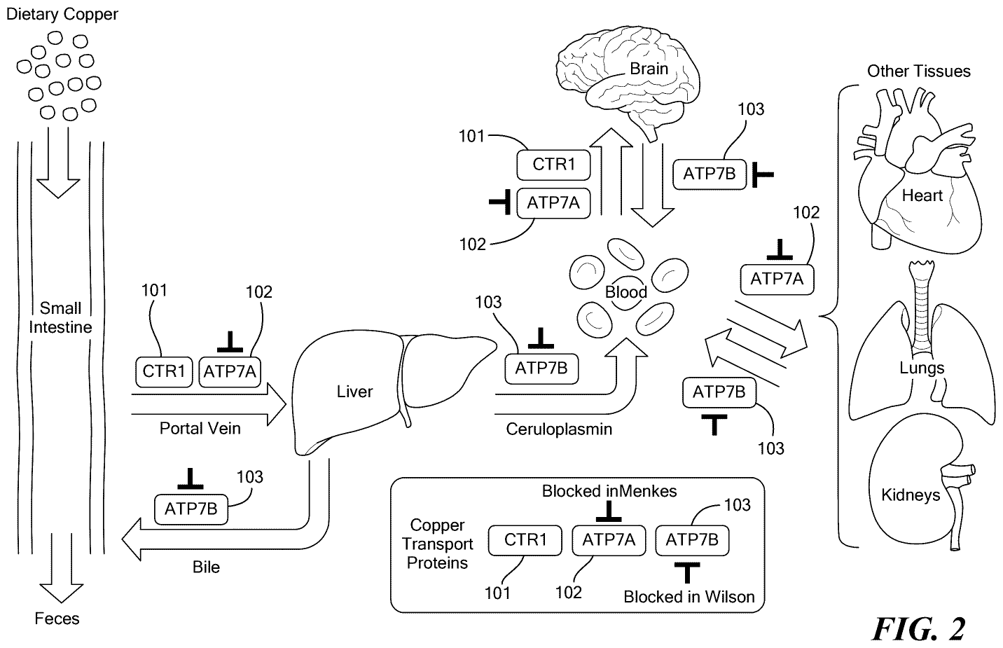

FIG. 2 shows an example of a therapeutic environment responsive to metal homeostasis regulation as defined herein;

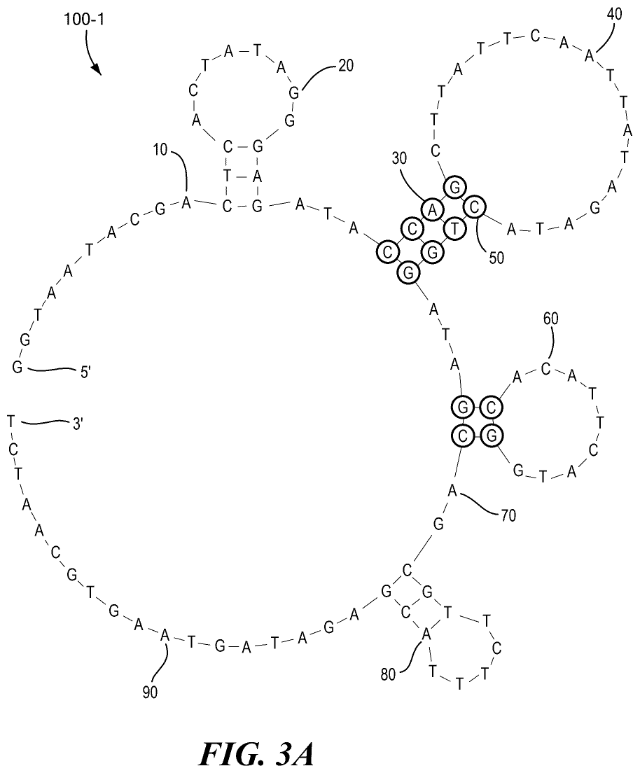

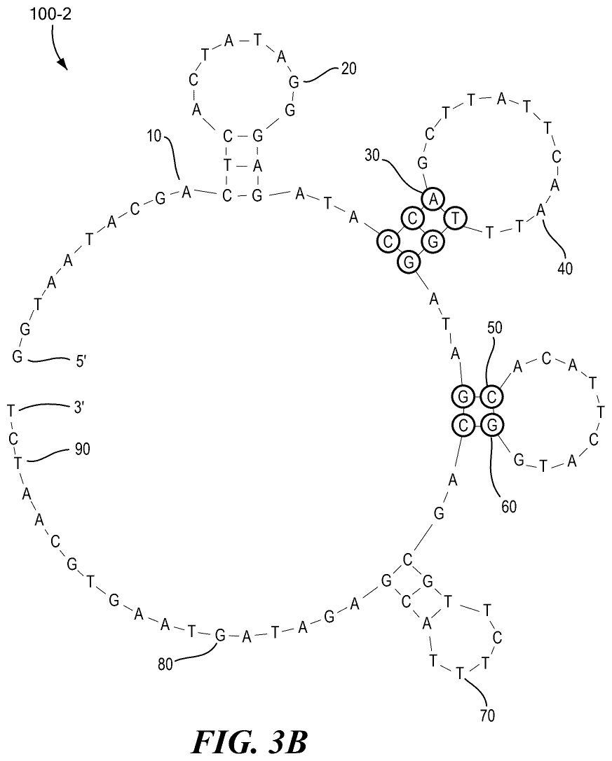

FIGS. 3A and 3B are variants of the binding aptamer of FIG. 1 suitable for use with configurations herein;

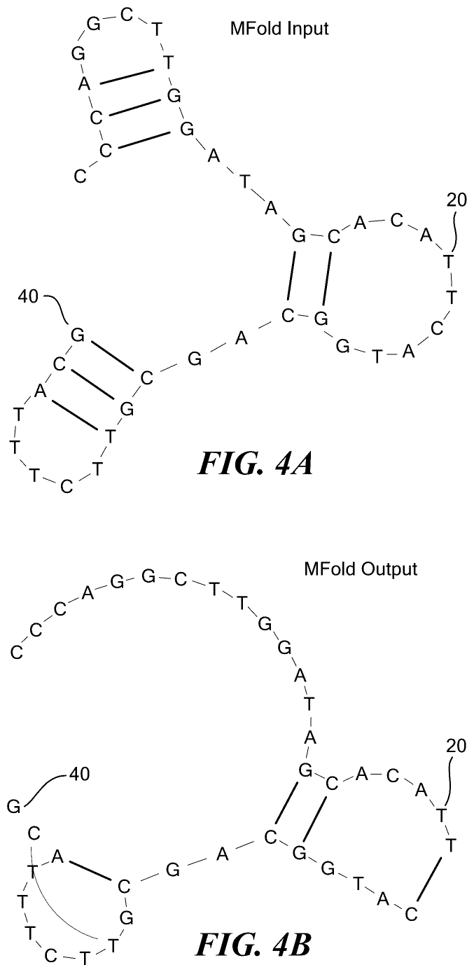

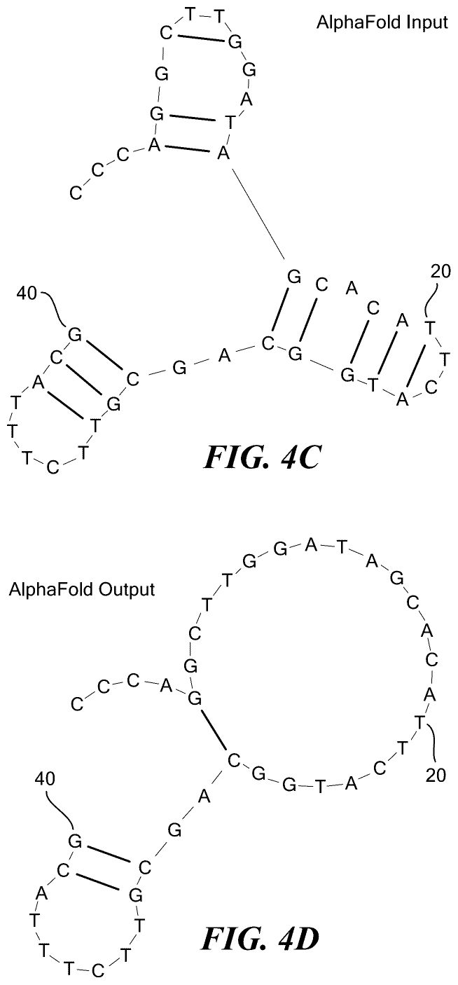

FIGS. 4A-4D show computed or predicted structures of binding aptamers for targeting trace metal ions as described herein;

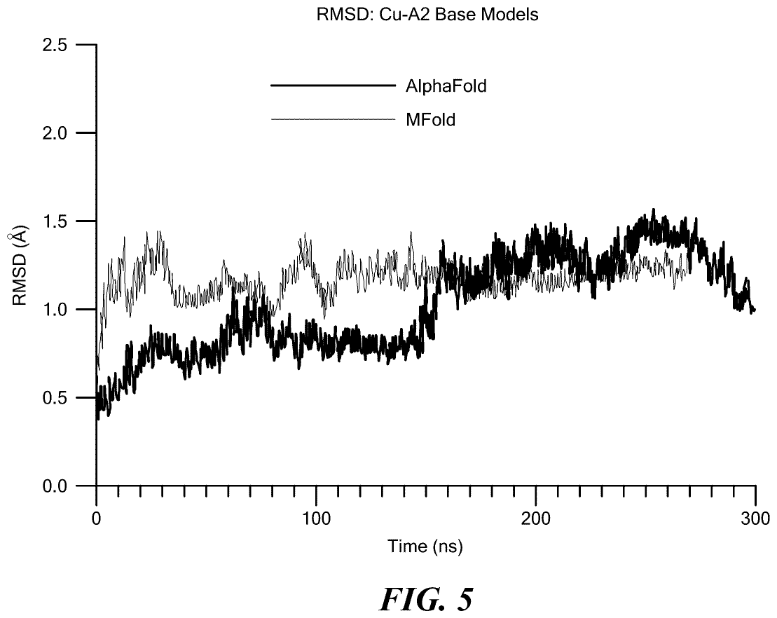

FIG. 5 shows a structural comparison of the binding aptamers of FIGS. 4A-4D;

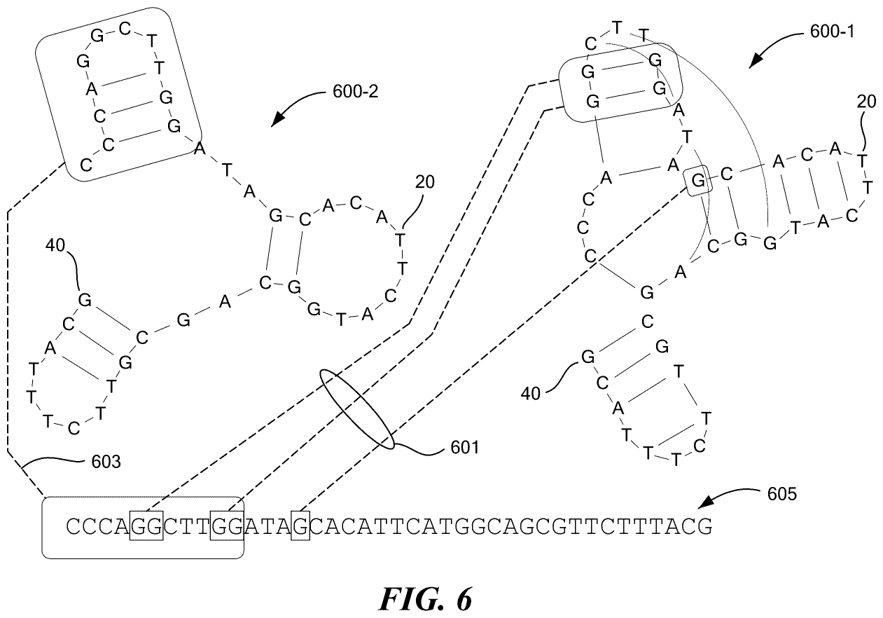

FIG. 6 shows binding interaction locations in the binding aptamers of FIGS. 4A and 4C; and

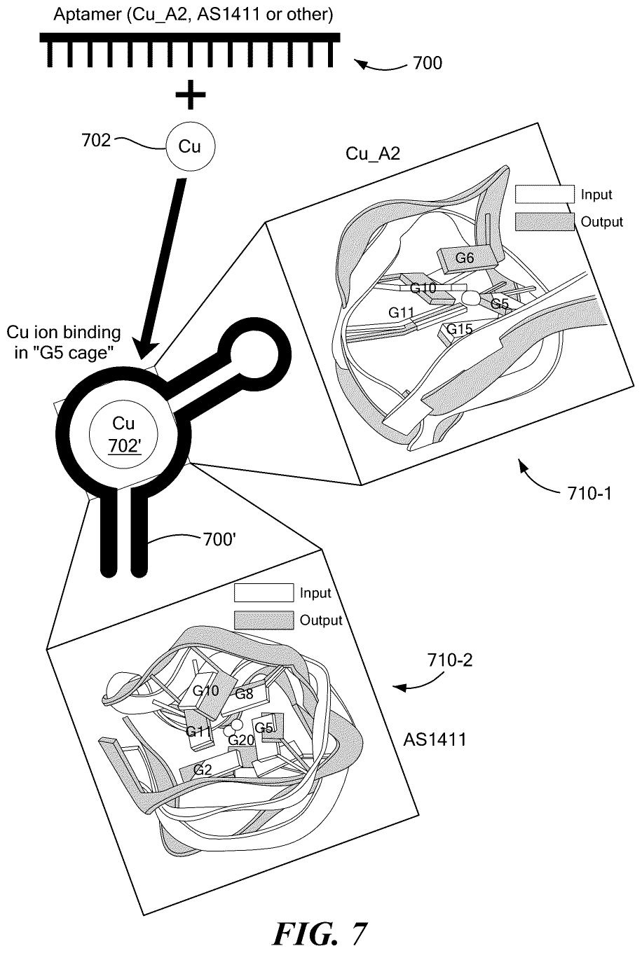

FIG. 7 shows a binding aptamer sequestering a trace metal via a structure forming the binding interaction locations for forming an electrostatic environment that favors the retention.

### DETAILED DESCRIPTION

Configurations described below present an approach to moderate or control the presence or absence of trace metals in the physiology of a biological specimen, typically human or animal. Trace elements (or trace metals) are minerals present in living tissues in small amounts. Some of them are known to be nutritionally essential, others may be essential, and still others are considered to be nonessential. Trace elements function primarily as catalysts in enzyme systems; for example, some metallic ions, such as iron and copper, participate in oxidation-reduction reactions in energy metabolism. Iron, as a constituent of hemoglobin and myoglobin, also plays a vital role in the transport of oxygen. Trace metals exist as ions, and are controlled by metal ion transporters in the subject specimen. As employed herein, a trace metal is a metal ion having a beneficial effect on physiology within concentrations of a predetermined range, and may have harmful effects at concentrations outside the predetermined range. The metal ion transporters are proteins that bind to the metal ions to promote a proper balance in the specimen. Certain diseases affect a balanced homeostasis due to a mutation affecting these transporter proteins, resulting in an improper concentration of the metal ions. For example, copper is known to be an “essential trace element”, as it is necessary for specific redox reactions and functions that occur in the human body, such as in the copper example above. When homeostasis fails due to inborn errors of metabolism, the result is copper metabolism diseases.

FIG. 1 is a diagram of a binding aptamer Cu-A2 100 having a sequence of nucleotides including binding interaction locations, as discussed further below, suitable for use with configurations herein. Conventional approaches to aptamer-metal interactions include treatment of heavy metal toxicity for eradicating tissue bound heavy metals, such as that disclosed in co-pending U.S. patent application Ser. No. 18/524,554, filed Nov. 30, 2023, entitled “HEAVY METAL TOXICITY REMEDIATION,” incorporated herein by reference in entirety and assigned to the assignee of the present application. In contrast to eradication of harmful molecules and compounds, the disclosed approach seeks homeostasis by controlled interaction of binding aptamers and bound metal ions to target specific tissue, organs or therapeutic regions to either promote or discourage concentrations of metal ions for maintaining a proper physiological range. Configurations herein first modify the nucleic acid to include the binding aptamer, and then introduce the modified nucleic acid including the binding aptamer into a therapeutic region in need of homeostasis regulation. In the example below, copper ions, specifically Cu²⁺, are the target trace metals. Other trace metals may include iron, zinc, manganese, selenium, lead, cadmium, mercury, arsenic and chromium.

Aptamers as employed herein refer to a short, single-stranded DNA or RNA molecule that can bind specifically to a target molecule, similar to how antibodies bind to antigens. These “artificial antibodies” are often generated through SELEX. Aptamers can bind to a wide variety of targets, including proteins, peptides, carbohydrates, and even cells.

Formation of a modified nucleic acid including the aptamer and a suitable biocompatible nucleotide strand may be performed according to several approaches. In a human or mammalian context, the delivery mechanism would deliver the aptamer in the form of DNA or RNA strand. The delivery mechanism to introduce the aptamer into the human physiology may be in the form of a capsule, therapeutic virus, probiotic bacteria, lipid nanoparticle, or other nanomaterials. The aptamer may be inside of the biocompatible delivery mechanism, or may be covalently or non-covalently attached to it. The aptamer may be released from the delivery mechanism or may remain within or bound to the delivery mechanism.

Configurations herein demonstrate aptamers that have the potential to bind to metal ions, including copper. One of the aptamers that has been found to selectively bind to copper ions includes the Cu-A2 DNA-aptamer. The Cu-A2 aptamer was found, to have the highest binding affinity of 1.83×10⁻¹¹ M among known candidates. The high binding affinity of copper to this aptamer may be a result of its overall structure as copper has been noted to bind preferentially to G-C pairs, and Cu-A2 has a structure that includes two stems of 5 G-C and a single stranded bridge, as seen in FIG. 1, which may facilitate the high binding affinity of the Cu-A2 aptamer, discussed further below. A further benefit is that the copper that was bound to the Cu-A2 aptamer could be recovered despite the high binding potential of the aptamer.

FIG. 2 shows an example of a therapeutic environment responsive to metal homeostasis regulation as defined herein. Two known diseases associated with copper metabolism imbalances are Wilson's disease and Menkes disease, which are normally caused by mutations in the ATP7B and ATP7A genes respectively. These genes encode for the ATP7A and ATP7B proteins respectively, which both function in copper transport.

Although over- and/or underconsumption of dietary copper may result in disease phenotypes, these cases are often easily treatable through dietary changes to rebalance the body's copper levels. In contrast, genetic mutations in vital copper transport proteins create larger systemic copper dysregulation and are much more difficult to treat. Menkes disease and Wilson's disease are two well-defined heritable diseases related to mutations in the copper transport proteins ATP7A and ATP7B respectively.

Referring to FIG. 2, In Menkes, loss of ATP7A function interferes with uptake of dietary copper into the blood, resulting in chronically low copper concentrations throughout the body. Menkes disease is characterized by neurodegeneration, connective tissue irregularities, and vascular abnormalities Due to the severity of symptoms in Menkes disease, patients have a very poor prognosis with a life expectancy of three years, although this may be shorter if not diagnosed and treated early enough. While other forms of copper deficiency can be more easily remedied through increased dietary copper intake, Menkes patients are unable to benefit from oral copper supplementation as uptake of dietary copper into the bloodstream is severely dampened. Intravenous copper administration using copper-histidine has shown some success in treating Menkes patients, but long term use can result in nephrotoxicity.

In contrast, loss of ATP7B function in Wilson's disease results in excess copper deposits in various tissues due to difficulty releasing excess copper. Similar to Menkes disease, Wilson's disease often presents with neurological symptoms due to the hyperactivity of copper-dependent proteins in the brain producing excessive reactive oxygen species. Wilson's disease also notably affects the liver, which is the primary storage location for excess copper. Since excess copper in the liver is unable to be exported into bile via ATP7B, copper often re-enters the circulatory system, resulting in further deposition in the brain and eyes. In severe cases, patients may develop liver disease, cirrhosis, or even liver failure. Additionally, upregulation of Ctrl and ATP7A in the presence of copper in cardiac tissue can result in pulmonary hypertension.

Systemic copper transport pathways shown in FIG. 2 include three primary copper transport proteins: CTR1 101, ATP7A 102, and ATP7B 103. CTR1 and ATP7A handle uptake of dietary copper and transport to the liver via the portal vein. ATP7B is responsible for copper export to other tissues via the blood and to bile for excretion. Import and export of copper ions are handled by ATP7A and ATP7B respectively. Disruption of transport pathways in Menkes and Wilson prevent copper homeostasis.

Accordingly, configurations herein present a method for metal homeostasis regulation such as in Wilson's and Menkes, including identifying a binding aptamer having an affinity for a trace metal, such as copper, and appending the binding aptamer to a nucleic acid to form a modified nucleic acid that also shares the affinity for copper. The modified nucleic acid is introduced into a therapeutic region for sequestering or introducing the trace metal via the affinity, and manipulation of the modified nucleic acid in the therapeutic region following sequestering of the trace metal allows the modified nucleic acid to mimic the operation of the repressed metal ion transport mechanism or protein and convey the copper accordingly through the physiology for achieving homeostasis.

Note that the disclosed approach is to form aptamers such as the Cu-A2 copper binding aptamer, and use them for two strategies for either increasing or decreasing copper ions:
1) inject or ingest aptamers (as modified nucleic acids) into individuals to bind metals in the body in the context of a metal overload disease and eliminate the metal ions from the body. The aptamer would sequester the metal ions and then be eliminated as a complex by urinary excretion. In some embodiments, the aptamers may be targeted to specific tissues by appending specific aptamer sequences that target tissues (e.g., an aptamer sequence that targets a receptor that is specific to the liver), or attaching additional chemical moieties of biomolecules including but not limited to glycosylations, antibodies, siRNAs, miRNAs to facilitate the targeting and absorption by different tissues.
2) Bind metals to aptamers prior to injection or ingestion, and have the binding properties of the aptamers deliver metals to particular tissues for targeted absorption, for the treatment of metal deficiencies or misdistribution. In some embodiments, this could involve tissue targeting with appended aptamer sequences or chemical modifications as described above. In this second strategy, binding first occurs with trace metal to the binding aptamer in the modified nucleic acid, and then introducing the modified nucleic acid into a therapeutic region for transporting the trace metal. Thus, homeostasis modulation may involve either increasing or decreasing the presence of a trace metal though nucleic acids including the binding aptamer.

Modified RNAs are increasingly being used as prophylactics and therapeutics in the form of mRNA vaccines, antisense oligonucleotides (ASOs), and small interfering (si) RNAs, and their impact is expected to increase rapidly and significantly. In one configuration, aptamers that specifically bind an individual metal could be injected into the bloodstream or tissues in a manner similar to current ASO and siRNA therapeutics, where it would circulate and bind to the target metal. Aptamers are known from human studies to be generally safe and efficiently excreted by the kidneys, which is a drawback for ASOs or siRNAs used for diseases, but in the context of eliminating metals may be an asset as the target metal ion would be efficiently eliminated with the aptamer in the kidney.

Configurations herein differ from conventional vaccines and therapeutics through the mechanism of action for a proposed treatment. In the case of aptamers, target binding interactions are most frequently attributed to the unique secondary structures formed by aptamers Nucleic acid secondary structure is thought to influence aptamer function through increased stability and target-binding affinity. The high flexibility of nucleic acid structures allows for the construction of numerous unique secondary structural motifs, such as stem loops, pseudoknots, and G-quadruplexes. These structures provide higher overall stability while also creating unique pockets for the binding of specific target molecules.

Configurations herein recognize the importance of secondary structure in aptamer behavior, as it was found that enrichment of SELEX libraries with higher secondary structure-forming sequences resulted in the production of higher affinity aptamers. In conjunction with the baseline functions of aptamer secondary structures, additional structure-switching can be present in aptamers. Structure-switching aptamers can be intentionally selected for or may arise through the standard SELEX process. Alterations in structure resulting from target-binding can modulate the function of the aptamer and typically strengthen the overall stability of the aptamer.

FIGS. 3A and 3B are variants of the binding aptamer of FIG. 1 suitable for use with configurations herein, and depicting secondary structure of aptamers and corresponding binding affinity. The secondary structures of Cu-A2-mutated (FIG. 3A, 100-1, Cu-A2Mut) and CU-A2-deleted (FIG. 3B, 100-2, Cu-A2Del) depict potential binding sites are highlighted with circular outlines and bold characters 301, as depicted in Table I.

TABLE I

| Aptamer | Sequence (5′ → 3′) | Source |
|---|---|---|
| (SEQ ID NO. 1) | CCCAGGCTTGGATAGCAC | (Sekhon et al., |
| Cu-A2 | ATTCATGGCAGCGTTCTT | 2017) |
| | TACG | |
| (SEQ ID NO. 2) | ATAGATACTGGATAGCAC | (Sekhon et al., |
| Cu-A2Mut | ATTCATGGCAGCGTTCTT | 2017) |
| | TACG | |
| (SEQ ID NO. 3) | TGGATAGCACATTCATGG | (Sekhon et al., |
| Cu-A2Del | CAGCGTTCTTTACG | 2017) |
| (SEQ ID NO. 4) | GATGTATAAGGCACCGGG | (Sekhon et al., |
| Cu-A2Ser | CTCCCGCTGTTTCCTTGC | 2017; Stothard, |
| | AATA | 2000) |
| (SEQ ID NO. 5) | GACGACCACGGTAAACGA | (Wang et al., |
| Cu-1 | CGCTGTACGGAGTGGTCT | 2024) |
| | GTCGTC | |
| (SEQ ID NO. 6) | GGTGGTGGTGGTTGTGGT | (Bahreyni et |
| AS1411 | GGTGGTGG | al., 2019) |

As structure and function are often inseparable concepts in biology, investigating the structure of copper-targeting aptamers may provide insight into the function of these aptamers. To that end, computational methods were applied to predict and describe copper-aptamer interactions. MD (Molecular Dynamics) simulations were performed for three copper-binding aptamers—Cu-A2, Cu-1, and AS1411—and their predicted structures, stabilities, and interaction energies when docked to copper ions were assessed. This was further broken down into three different tests. First, two base model construction strategies—MFold and AlphaFold—were compared using the Cu-A2 aptamer to see which method would produce higher stability, lower energy models. This test was performed both in the presence and absence of copper, and it was used to determine the modeling method to apply to all other aptamers moving forward. The same MD experiment was then conducted using the Cu-A2 mutants, Cu-A2Mut, Cu-A2Del, and Cu-A2Scr to assess how structural variation influences copper-aptamer interactions. This test also served to further validate this methodology, as these mutant aptamers are known to have reduced copper binding affinity. Finally, this pipeline was applied to the two alternative copper-binding aptamers, Cu-1 and AS1411, to determine whether these aptamers also showed strong copper-binding activity and to identify key structures associated with copper-aptamer binding. From this, a common “guanine-cage”formation was identified in Cu-A2 and AS1411 that allowed highly energetically favorable interactions with copper. Circular dichroism (CD) spectroscopy was performed to validate the predicted interaction between AS1411 and copper. As described further below, this guanine cage formed from the binding aptamer further defines the binding interaction locations on the aptamer, such that the binding interaction location is defined by a nucleotide forming an electrostatic affinity for the trace metal. As discussed further below with respect to FIGS. 6 and 7, the G (guanine sites) do not necessarily need to interact with each other, but rather create an electrostatic environment that favors the retention of a copper (Cu²⁺) ion.

FIGS. 4A-4D show computed or predicted structures of binding aptamers for targeting trace metal ions as described herein. The disclosed example configuration employs an MD simulation commencing with construction of a base model of the molecule of interest. Currently, there is no standardized approach to computationally generating 3D DNA structures. However, two methods, which will be referred to as the MFold and AlphaFold methods, are common for structure prediction. The MFold method uses the MFold program to generate a 2D secondary structure prediction for a given sequence. The resulting sequence is fed into RNAComposer, which constructs a 3D RNA model. When working with DNA, this RNA model has to be mutated back to DNA. The AlphaFold method allows for full 3D DNA model construction from a DNA sequence through sequence input in AlphaFold3.

FIG. 4A-4D show predicted structures of the Cu-A2 aptamer before and after simulation. The MFold model (FIGS. 4A-4B) was generated using MFold and RNAComposer. The AlphaFold model (FIGS. 4C-4D) was generated using the AlphaFold3 web server. Both predicted structures were run through 300 ns of simulation in GROMACS, and the final 2D secondary structures were constructed based on hydrogen bonding in the 3D models, as depicted in FIG. 4B (MFold) and FIG. 4D (AlphaFold).

In order to ensure use of the best possible predicted base models for MD simulation, models of the Cu-A2 aptamer using both methods were generated, and 300 ns simulations were run to assess their behavior. Both base models started with a fairly similar three-loop structure, however the AlphaFold model appears to be more spatially compact. After 300 ns of simulation, both structures ended with only two loops, and the overall structure loosened in both cases via widespread base fraying.

FIG. 5 compares the RMSD of Cu-A2 mutant aptamers of FIGS. 4A-4D. The RMSD (Root Mean Square Deviation) of each model over time indicated similar distances from the starting structures, indicating that the initial predictions in both models were of similar accuracy to the most stable conformation. Both models appear to reach stability at around 40 ns, the average RMSD for the predicted models are 1.18±0.09 Å and 1.02±0.27 Å for the MFold and AlphaFold models respectively, as confirmed in FIG. 5. RMSD is a measure of the average distance between the atoms of two superimposed protein structures. It is an established metric used to assess structural similarity and is particularly useful in protein structure prediction and molecular dynamics simulations.

Since the base model predictions from both methods (AlphaFold and MFold) were comparable in both overall structure and behavior over the simulation, it was further examined whether there were differences in how these two methods predicted copper binding interactions. Two docked models were generated for each method, and all models represented Cu-A2 binding to copper ions. To facilitate identifying the binding aptamer having an affinity or attraction for the trace metal, it is beneficial to identify a binding interaction location on the aptamer, the binding interaction location defined by a nucleotide forming an electrostatic affinity for the trace metal.

FIG. 6 shows binding interaction locations in the binding aptamers of FIGS. 4A and 4C. Referring to FIGS. 4A-4D and 6, binding interaction locations 601 of a binding aptamer 600-1 are shown for guanine bases (AlphaFold, right) and binding interaction locations 603 for the MFold base (left) depict a binding aptamer 600-2 (600 generally) with copper ion placement relative to positions in the Cu-A2 strand 605.

In the MFold model, a copper ion was added to both the original base model prediction (MFold-1) and to the final structure of the base model after 300 ns of simulation (MFold-2). The placement of the copper ion was chosen based on the first loop as a potential key binding pocket, shown in FIG. 6, right. Within this first loop, there are four different guanine bases that may be involved in copper-binding interactions: G5, G6, G10, and G11, defining the binding interaction locations 601. However, not all of these bases were in close proximity in both the starting and final MFold model, so ion binding locations were limited. For MFold-1, the copper ion was placed between G10 and G11, with the closest contact point to each base after equilibration being 2.895 Å and 3.491 Å respectively. For MFold-2, the copper ion was placed between G6 and G10, as these were the only pair of bases from the original four predicted bases that were in close proximity to one another. After equilibration, the closest contact point to each base from the copper ion was 3.111 Å to G6 and 3.960 Å to G10.

In the AlphaFold model, AlphaFold3 was first used to create a docked model by inputting both the Cu-A2 aptamer sequence and a copper ion. The resulting prediction, AlphaFold-1, maintained a very similar three-loop structure, this time containing a psuedoknot connecting the first two loops, demonstrated in FIG. 6, left. This pseudoknot results in a pocket containing all four predicted guanine bases as well as a fifth guanine, G15. After equilibration, the nearest contact point from copper to each base was 2.026 Å to G5, 2.091 Å to G6, 1.777 Å to G10, 1.827 Å to G11, and 2.031 Å to G15. To verify that copper binding to these bases was specific, an additional model, AlphaFold-2, was generated taking the first AlphaFold docking model and moving the copper ion to a different G-C rich region. The new ion placement was closest to C1, G29, C30, and G40, depicting the binding interaction locations 603.

FIG. 6 therefore demonstrates that the binding aptamer has a plurality of binding interaction locations, where a respective nucleotide base defining each of the binding interaction locations, such that the plurality of binding interaction locations are drawn to the trace metal for forming the binding aptamer into a shape surrounding the trace metal.

FIG. 7 shows a binding aptamer sequestering the trace metal copper via a structure forming the binding interaction locations for forming an electrostatic environment that favors the retention using 5 binding interaction locations. FIG. 7 depicts a binding aptamer 700 of Cu-A2 or AS1411 and a trace metal of copper 702, and the binding interaction locations formed from guanine to define the “cage” structure 710-1 . . . 710-2 (710 generally) of the aptamer around the copper ion. Once a suitable aptamer 700 is identified, the delivering nucleic acid is modified to include the aptamer capable of binding (in the case of trace metal removal) or bound to the trace metal (in the case of trace metal augmentation). In the case of trace metal augmentation or delivery, it is important that an affinity of the trace metal in the deficient therapeutic region is sufficient to overcome an affinity of the trace metal to the modified nucleic acid, so that once the trace metal is delivered it may unbind to freely interact with the physiology of the recipient . . . . FIG. 7 is a graphical rendering of the cage 710-1 . . . 710-2 (710 generally) that forms around the trace metal from the binding interaction locations. Referring to FIGS. 6 and 7, the selected aptamer 700 is known to bind to a particular trace metal 702, copper in the example shown, to form a bound copper ion 702′.

It is noteworthy that the binding interaction locations are all guanine bases on the binding aptamer sequence for forming the cage 710. The guanine base positions define an electrostatic environment favoring retention of the trace metal. The bound aptamer 700′ forms the cage 710 from the binding aptamer based on a secondary structure defined by five binding interaction locations forming a binding pocket around a copper ion. In a particular configuration, the binding interaction locations include G5, G6, G10, G11, and G15 on the Cu-A2 aptamer 710-1. In another example, the binding interaction locations include G5, G8, G10, G11, and G20 on the AS1411 aptamer 710-2. Conventional approaches have not shown that 5 guanine nucleotides (rather than 4, as in known G-quadruplex structures) are significant for efficient binding to copper ions. It does not matter specifically where within the sequence the five Gs are, but only that they are capable of folding together to surround the copper ion. The Gs do not interact with each other, but rather create an electrostatic environment that favors the retention of a copper (Cu²⁺) ion.

Once the base model prediction method was selected, as described above comparisons of different Cu-A2 mutant aptamers were conducted, as shown in Table I. The Cu-A2Mut and Cu-A2Del aptamers were previously shown to have a lower copper binding affinity, likely resulting from loss of bases G5, and G6. Additionally, the Cu-A2Scr aptamer is not expected to interact favorably with copper ions. Models of all Cu-A2 mutants are shown in Table I. The Cu-A2Mut docking model has the most bases in close proximity to the copper ion out of the three mutant aptamers. The closest contact points to each base to the copper ion in Cu-A2Mut are 2.910 Å to G10, 1.968 Å to G11, and 3.115 Å to C30. This docking position is somewhat similar to that of the original Cu-A2 aptamer, as Cu-A2Mut retains contacts to G10 and G11, but loss of bases G5 and G6 renders Cu-A2Mut incapable of reforming the full Cu-A2 binding pocket. Cu-A2Del also shares similar interactions, though the numbering is slightly different due to deletion of the first 6 nucleotides. The closest nucleotides to Cu-A2Del are G2 and C22, which are analogous to G10 and C30 in Cu-A2 and Cu-A2Mut. The closest contact points from the copper ion to G2 and C22 are 1.810 Å and 3.088 Å respectively. In the docking model for the final mutant aptamer, Cu-A2Scr, the copper ion is only in close proximity to a singular base, G11, with its closest contact point being 3.403 Å from G11. These three mutant aptamers stabilized within the first 40 ns of simulation, and they all had higher average RMSD values, those being 1.16±0.13 in Cu-A2Mut, 1.23±0.24 in Cu-A2Del, and 1.16±0.15 in Cu-A2Scr. These higher RMSD values indicate a greater degree of structural change from their base models than that of the original Cu-A2 aptamer.

The relationship between structure and function is a key component of the biomolecular interactions discussed herein. Accordingly, configurations of FIGS. 6 and 7 seek to determine how aptamer structure affects copper binding. Though the tools available for DNA structural prediction are limited in comparison to those available for proteins, the disclosed approach utilizes a computational modeling pipeline for the assessment of aptamer-target interactions. The base models that resulted the most energetically favorable docking site predictions were generated using the AlphaFold3 server, so all AlphaFold docking models were prepared for each aptamer of interest as inputs for MD simulations. In simulating Cu-A2 and its mutants—Cu-A2Mut, Cu-A2Del, and Cu-A2Scr—a loss of favorable copper-aptamer interactions was observed as demonstrated by the increased interaction energy in the mutants. In Cu-A2Mut and Cu-A2Del, where two of the bases responsible for binding in Cu-A2 are no longer present, the models showed short periods of loose copper-aptamer interactions followed by periods of ion dissociation. This seems to correlate with a decrease, but not a total loss, of copper binding affinity in Cu-A2Mut and Cu-A2Del. Alternatively, Cu-A2Scr was observed to have little to no interaction with the copper ion across the full simulation, likely as a result of the complete rearrangement of the predicted binding pocket. In simulations of two additional copper-targeting aptamers, Cu-1 and AS1411, AS1411 was shown to have remarkably similar behavior to Cu-A2. Both the Cu-A2 and AS1411 binding pockets are defined by a cage of five guanines surrounding the copper ion. In both simulations, the guanine cage holds the copper ion in place, resulting in high stability at the binding pocket and a very low interaction energy (−1430.40±41.51 KJ/mol in Cu-A2 and −1426.10±38.57 KJ/mol in AS1411). AS1411 binding to copper was subsequently verified via CD spectroscopy, which showed a large shift in the CD spectrum when exposed to 50 uM copper sulfate.

The five-guanine cage structure observed in both Cu-A2 and AS1411 docked models indicates a preferred binding structure for copper ions. Copper is already known to interact with guanine bases via G-C chelates and G-G sandwiches. Prior data pertaining to copper-DNA interactions would indicate that copper ions would preferentially bind to stacked guanines and/or G-C base pairs; however, the dissociation of copper ions in nearly all models despite the presence of numerous G-C base pairs suggests that G-C base pairs alone may not be the sole association to generate a high binding affinity. On the contrary, the significantly lower interaction energy observed in both Cu-A2 and AS1411, which both have guanine cages, does not seem coincidental. This common structure aligning with similar behavior suggests that the five-guanine cage may be significant for generating high-affinity copper-targeting aptamers.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for metal homeostasis regulation, comprising:
identifying a binding aptamer having an affinity for a trace metal;
appending the binding aptamer to a nucleic acid to form a modified nucleic acid;
introducing the modified nucleic acid into a therapeutic region for sequestering the trace metal; and
removing the modified nucleic acid from the therapeutic region following sequestering of the trace metal.

2. The method of claim 1 wherein identifying the binding aptamer further comprises identifying a binding interaction location on the aptamer, the binding interaction location defined by a nucleotide forming an electrostatic affinity for the trace metal.

3. The method of claim 1 wherein the binding aptamer has a plurality of binding interaction locations, further comprising identifying a respective nucleotide base defining each of the binding interaction locations, the plurality of binding interaction locations drawn to the trace metal for forming the binding aptamer into a shape surrounding the trace metal.

4. The method of claim 3 wherein the binding interaction locations are guanine bases on the binding aptamer sequence.

5. The method of claim 3 wherein the binding interaction locations include G5, G6, G10, G11, and G15.

6. The method of claim 3 wherein the binding interaction locations include G5, G8, G10, G11, and G20.

7. The method of claim 3 wherein the binding aptamer is Cu-A2 or AS1411 and the trace metal is copper.

8. The method of claim 1 wherein the trace metal is one or more of iron, zinc, manganese, selenium, lead, cadmium, mercury, arsenic or chromium.

9. The method of claim 1 further comprising:
modifying the nucleic acid to include the binding aptamer; and
introducing the modified nucleic acid including the binding aptamer into a therapeutic region in need of homeostasis regulation.

10. The method of claim 1 further comprising:
identifying deficient therapeutic region having an insufficient concentration of the trace metal;
identifying a nucleic acid having an affinity for the deficient therapeutic region or tissue common to the deficient therapeutic region; and
modifying the nucleic acid to include the aptamer bound to the trace metal.

11. The method of claim 10 wherein an affinity of the trace metal in the deficient therapeutic region is sufficient to overcome an affinity of the trace metal to the modified nucleic acid.

12. The method of claim 10 further comprising appending a targeting aptamer to the modified nucleic acid, the targeting aptamer selected based on an affinity for the deficient therapeutic region.

13. The method of claim 1 wherein the trace metal is a metal ion having a beneficial effect on physiology within concentrations of a predetermined range, and harmful effects at concentrations outside the predetermined range.

14. The method of claim 3 wherein the guanine base positions define an electrostatic environment favoring retention of the trace metal.

15. The method of claim 3 further comprising forming a cage from the binding aptamer based on a secondary structure defined by five binding interaction locations forming a binding pocket around a copper ion.

16. A method for metal homeostasis regulation, comprising:
identifying a binding aptamer having an affinity for a trace metal;
appending the binding aptamer to a nucleic acid to form a modified nucleic acid;
binding the trace metal to the binding aptamer in the modified nucleic acid; and
introducing the modified nucleic acid into a therapeutic region for transporting the trace metal.

17. The method of claim 16 further comprising:
selecting the nucleic acid based on an affinity or tendency to a therapeutic region.

18. The method of claim 16 wherein the binding aptamer is Cu-A2 and the trace metal is a copper ion.
